(19) United States
(12) Patent Application Publication
(10) Pub. No.: US 2026/0215784 A1
(43) Pub. Date: Jul. 30, 2026

(54) INTRAVASCULAR FLOW DIVERTER AND RELATED METHODS

(71) Applicant: NV MEDTECH, Inc., Carson City, NV (US)

(72) Inventors: Michael Jones, Gardnerville, NV (US); Thomas James Mock, Reno, NV (US); Joseph Emery, Carson City, NV (US); Ibraheem Khan, Reno, NV (US); Kenna Ryan, Reno, NV (US)

(21) Appl. No.: 19/572,852

(22) Filed: Mar. 19, 2026

Related U.S. Application Data
(63) Continuation-in-part of application No. 18/631,948, filed on Apr. 10, 2024.
Application No. 18/631,948 is a continuation of application No. 18/113,465, filed on Feb. 23, 2023, now Pat. No. 11,957,356.
(63) Continuation-in-part of application No. 18/113,493, filed on Feb. 23, 2023.
(60) Provisional application No. 63/313,487, filed on Feb. 24, 2022.
(60) Provisional application No. 63/313,635, filed on Feb. 24, 2022.
(60) Provisional application No. 63/943,719, filed on Dec. 18, 2025.

(51) Int. Cl. A61B 17/12

(52) U.S. Cl. CPC A61B 17/12145; A61B 17/12113; A61B 17/12118; A61B 2017/1205

(57) ABSTRACT

An intravascular flow diverter for treating an aneurysm of a blood vessel is provided. The intravascular flow diverter includes a frame comprising a plurality of wires forming an expandable structure. The intravascular flow diverter includes a cover disposed on at least a portion of the outer surface of the device. The cover includes a first portion configured to be disposed directly against a neck of the aneurysm.

### CROSS REFERENCE TO RELATED APPLICATIONS

The present application is a continuation-in-part of U.S. patent application Ser. No. 18/631,948, which application is a continuation of U.S. application Ser. No. 18/113,465, which application claims priority to U.S. Provisional Application 63/313,487. The present application is also a continuation-in-part of U.S. patent application Ser. No. 18/113,493, which application claims priority to U.S. Provisional Application 63/313,635. The present application also claims priority to U.S. Provisional Application 63/943,719.

### BACKGROUND

Intracranial aneurysms are among the most serious of medical conditions. Their typical size and location make them especially difficult to detect and treat; but even small ones, if ruptured, can cause debilitating physical and cognitive impairment, coma, and death. Initial treatment methods involved clip ligation of the neck of the aneurysm in open surgical procedures. More recently, minimally invasive endovascular techniques have been developed. Given the clinical significance of the condition and the difficulties encountered in addressing it, treatment for intracranial aneurysms remains an especially active area of device and surgical procedure development.

Currently, the Medtronic Pipeline™ flow diverter is a widely used device for flow diversion for aneurysm treatment. The device typically has 48 to 64 wires formed into an open braid with a metal coverage of the sidewall defined by the braid of about 30-50% when the device is deployed. The pore sizes bounded by the metal braid for the Pipeline may have more than 100 micron areal equivalent diameter.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

In one embodiment, an intravascular flow diverter for treating an aneurysm of a blood vessel comprises one or more wires forming a frame, wherein the frame has a collapsed configuration and an expanded configuration, wherein the frame is configured to transition in use from the collapsed configuration to a deployed configuration that substantially conforms to a shape of an inside surface of the blood vessel in the vicinity of the aneurysm. A cover is coupled to and extends over at least a portion of the frame and comprises pores therethrough over the at least a portion of the frame. In the expanded configuration over at least 70% of the total cover area: (1) a thickness of the cover is less than 7 microns, (2) less than 5% of the pores have an areal equivalent pore diameter of 20 microns or more, (3) a median areal equivalent pore diameter is between 3 and 9 microns, (4) 10% to 70% of the pores by pore count have an areal equivalent pore diameter of less than 5 microns, (5) 5% to 30% of the pores by pore count have an areal equivalent pore diameter of 10 microns or more, and (6) the flow diverter surface coverage over the at least 70% of the total cover area is between 45% and 85%.

In another embodiment, an intravascular flow diverter for treating an aneurysm of a blood vessel comprises one or more wires forming a frame, wherein the frame has a collapsed configuration and an expanded configuration, wherein the frame is configured to transition in use from the collapsed configuration to a deployed configuration that substantially conforms to a shape of an inside surface of the blood vessel in the vicinity of the aneurysm. A cover is coupled to and extends over at least a portion of the frame and the cover comprises pores therethrough over the at least a portion of the frame. In the expanded configuration (1) a thickness of the cover is less than 7 microns, (2) at least 95% of the pores by pore count have an areal equivalent pore diameter of less than 20 microns, (3) 20% to 45% of the pores by pore count over the at least 70% of the total cover area have an areal equivalent pore diameter of less than 3 microns, and (4) a median areal equivalent pore diameter of the pores is less than 5 microns.

In another embodiment, an intravascular flow diverter for treating an aneurysm of a blood vessel comprises one or more wires forming a frame, wherein the frame has a collapsed configuration and an expanded configuration, wherein the frame is configured to transition in use from the collapsed configuration to a deployed configuration that substantially conforms to a shape of an inside surface of the blood vessel in the vicinity of the aneurysm. A cover is coupled to and extends over at least a portion of the frame and the cover comprises pores therethrough over the at least a portion of the frame. In the expanded configuration (1) a thickness of the cover is less than 7 microns, (2) at least 95% of the pores by pore count have an areal equivalent pore diameter of less than 20 microns, (3) 20% to 30% have an areal equivalent pore diameter of 10 or more microns, and (4) wherein a median areal equivalent pore diameter over the at least 70% of the total cover area is between 6 and 9 microns.

In another embodiment, a surgical kit comprises a first plurality of flow diverters and a second plurality of flow diverters. The two pluralities of flow diverters have different pore characteristics such as median pore size and are segregated in the kit so the surgeon can select which flow diverter to use for a procedure based on the different pore characteristics and the nature of the procedure.

In some embodiments, such a surgical kit comprises a first plurality of flow diverters, wherein (1) each flow diverter of the first plurality of flow diverters comprises one or more wires forming a frame, wherein the frame has a collapsed configuration and an expanded configuration, wherein the frame is configured to transition in use from the collapsed configuration to a deployed configuration that substantially conforms to a shape of an inside surface of an intracranial blood vessel in the vicinity of an aneurysm, (2) each flow diverter of the first plurality of flow diverters comprises a cover comprising electrospun filaments coupled to and extending over at least a portion of the frame, (3) the cover for each flow diverter of the first plurality of flow diverters comprises pores therethrough over the at least a portion of the frame, (4) the pores define a median areal equivalent pore diameter for each one of the first plurality of flow diverters, and (5) a range of median areal equivalent pore diameters over the first plurality of flow diverters is 2 microns or less. The kit further includes a second plurality of flow diverters, wherein (1) each flow diverter of the second plurality of flow diverters comprises one or more wires forming a frame, wherein the frame has a collapsed configuration and an expanded configuration, wherein the frame is configured to transition in use from the collapsed configuration to a deployed configuration that substantially conforms to a shape of an inside surface of an intracranial blood vessel in the vicinity of an aneurysm, (2) each flow diverter of the second plurality of flow diverters comprises a cover comprising electrospun filaments coupled to and extending over at least a portion of the frame, (3) the cover for each flow diverter of the second plurality of flow diverters comprises pores therethrough over the at least a portion of the frame, (4) the pores define a median areal equivalent pore diameter for each one of the second plurality of flow diverters, and (5) a range of median areal equivalent pore diameters over the second plurality of flow diverters is 2 microns or less. The upper endpoint of the range of median areal equivalent pore diameters over the first plurality of flow diverters is separated from the lower endpoint of the range of medial areal equivalent pore diameters over the second plurality of flow diverters by at least 1 micron, and the first plurality of flow diverters and the second plurality of flow diverters are segregated from one another in the surgical kit such that a user of the kit can selectively deploy a flow diverter from the first plurality flow diverters or the second plurality of flow diverters to treat an aneurysm.

In some of these kits, in the expanded configuration a thickness of the covers of the first plurality of flow diverters and the second plurality of flow diverters over at least 70% of the total cover area is less than 10 microns or less than 7 microns.

In some of these kits, the intravascular flow diverters of the first plurality of flow diverters and the second plurality of flow diverters are provided in size increments in the expanded configuration of 0.5 mm or more, or 1.0 mm or more, or more than 1.0 mm.

In some of these kits, the upper endpoint of the range of median areal equivalent pore diameters over the first plurality of flow diverters is separated from the lower endpoint of the range of medial areal equivalent pore diameters over the second plurality of flow diverters by at least 2 microns.

In some of these kits, the upper endpoint of the range of median areal equivalent pore diameters of the first plurality of flow diverters is less than 5 microns, and in some the lower endpoint of the range of median areal equivalent pore diameters of the second plurality of flow diverters is more than 7 microns.

In some such kits, a third plurality of flow diverters are provided, wherein (1) each flow diverter of the third plurality of flow diverters comprises one or more wires forming a frame, wherein the frame has a collapsed configuration and an expanded configuration, wherein the frame is configured to transition in use from the collapsed configuration to a deployed configuration that substantially conforms to a shape of an inside surface of an intracranial blood vessel in the vicinity of an aneurysm, (2) each flow diverter of the third plurality of flow diverters comprises a cover comprising electrospun filaments coupled to and extending over at least a portion of the frame, (3) the cover for each flow diverter of the third plurality of flow diverters comprises pores therethrough over the at least a portion of the frame, (4) the pores define a median areal equivalent pore diameter for each one of the third plurality of flow diverters, and (5) a range of median areal equivalent pore diameters over the third plurality of flow diverters is 2 microns or less. The upper endpoint of the range of median areal equivalent pore diameters over the second plurality of flow diverters is separated from the lower endpoint of the range of medial areal equivalent pore diameters over the third plurality of flow diverters by at least 1 micron, and the first plurality of flow diverters and the second plurality of flow diverters and the third plurality of flow diverters are segregated from one another in the surgical kit such that a surgeon using the kit can selectively deploy a flow diverter from the first plurality or the second plurality or the third plurality to treat an aneurysm.

In some such kits, some or all of the flow diverters are pre-loaded in microcatheters for surgical deployment.

In another embodiment., an intravascular flow diverter for treating an aneurysm of a blood vessel comprises one or more wires forming a frame, wherein the frame has a collapsed configuration and an expanded configuration, wherein the frame is configured to transition in use from the collapsed configuration to a deployed configuration that substantially conforms to a shape of an inside surface of the blood vessel in the vicinity of the aneurysm, a cover coupled to and extending over at least a portion of the frame, wherein the cover comprises pores therethrough over the at least a portion of the frame. In the expanded configuration a thickness of the cover over at least 70% of the total cover area is less than 7 microns. In the expanded configuration at least 95% of the pores by pore count over the at least 70% of the total cover area have an areal equivalent pore diameter of less than 20 microns. In the expanded configuration 5% to 20% of the pores by pore count over the at least 70% of the total cover area have an areal equivalent pore diameter of 12 or more microns, and in the expanded configuration a median areal equivalent pore diameter of the pores over the at least 70% of the total cover area is between 3 and 9 microns.

In another embodiment, a method of treating a main vessel sidewall aneurysm, wherein the main vessel has at least one perforating branch vessel adjacent to the sidewall aneurysm. The method comprises expanding a flow diverter within the main vessel and against the sidewall of the main vessel to a deployed configuration over a neck of the sidewall aneurysm and over an entry opening of the perforating branch vessel. The flow diverter comprises a cover, wherein the cover comprises pores therethrough, wherein a first portion of the cover is positioned over the neck of the sidewall aneurysm and a second portion of the cover is positioned over the entry opening of the perforating branch vessel, and wherein a median areal equivalent diameter of the pores of the first portion and a median areal equivalent diameter of the pores of the second portion are between 3 and 9 microns. In some of these methods, at least 95% of the pores by pore count of the first portion of the cover and the second portion of the cover have an areal equivalent pore diameter of less than 20 microns. In some of these methods, 5% to 20% of the pores by pore count of the first portion of the cover and the second portion of the cover have an areal equivalent pore diameter of 12 or more microns. In some of these methods, the first portion of the cover and the second portion of the cover consist essentially of electrospun filaments. In some of these methods, a thickness of the cover over at least 70% of the total cover area is less than 7 microns.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIG. 1A illustrates a portion of a blood vessel having an aneurysm, in accordance with some embodiments;

FIG. 1B illustrates an intravascular flow diverter located across the neck of an aneurysm.

FIG. 2 illustrates a microcatheter disposed within the blood vessel and near the aneurysm, in accordance with some embodiments;

FIG. 3 illustrates an intravascular flow diverter disposed within the blood vessel and immediately against the aneurysm, in accordance with some embodiments;

FIG. 4 illustrates a magnified view of a portion of the intravascular flow diverter of FIG. 3, in accordance with some example embodiments;

FIG. 5A illustrates a cross-section of a first embodiment of the intravascular flow diverter of FIG. 4, in accordance with some example embodiments;

FIG. 5B illustrates a cross-section of a second embodiment of the intravascular flow diverter of FIG. 4, in accordance with some example embodiments;

FIG. 5C illustrates a cross-section of a third embodiment of the intravascular flow diverter of FIG. 4, in accordance with some example embodiments;

FIG. 6 is a magnified image of a portion of a flow diverter according to some embodiments;

FIGS. 7A and 7B are histograms of areal equivalent pore diameters for example flow diverters in accordance with some embodiments;

FIG. 8A is a graph of model comparative test results for perforator pressure drops;

FIG. 8B is a graph of model comparative test results for parent vessel flow changes;

FIG. 9 illustrates a section of vasculature with a deployed flow diverter according to some embodiments;

FIG. 10A illustrates a section of vasculature containing an aneurysm and adjacent perforator vessels prior to deployment of a flow diverter;

FIG. 10B illustrates the section of vasculature of FIG. 10A after deployment of a flow diverter according to embodiments of the invention;

FIGS. 11A and 11B illustrate endothelialization of a deployed flow diverter according to some embodiments; and

FIGS. 12A and 12B also illustrate endothelialization of a deployed flow diverter according to some embodiments.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

### Definitions

Collapsed Configuration—A device is in a collapsed configuration when it is sheathed proximate to and inside a distal end of a catheter ready for use in an endovascular surgical procedure.
Expanded Configuration—A device is in an expanded configuration when unsheathed outside the vasculature such that outward expansion of the sidewall is unconstrained by any surrounding walls. For flow diverters that are manually expanded by the surgeon, the expanded configuration is obtained when the flow diverter is manually expanded to its maximum intended diameter for normal use.
Deployed Configuration—A device is in the deployed configuration when unsheathed and with its side wall in contact with the inner wall of a vessel. A deployed configuration may have a smaller diameter for the sidewall than an expanded configuration depending on the size of the vessel in which the device is deployed. A device in the deployed configuration is typically close to but not fully in the expanded configuration, such as, a deployed diameter may be between 75% and 90% of an expanded diameter. Where the deployed configuration is being defined outside of a vessel, it may be defined as a device diameter of 80% of the device diameter in the expanded configuration.
Frame—One or more struts forming a structural scaffold defining a device sidewall configured to conform to the inner surface of a vessel segment when the device is in a deployed configuration. An arrangement of metal wires is a common implementation of struts for a frame.
Frame Porosity—The fractional or equivalent percent open area of a selected portion of the sidewall defined by the struts of the device when the device is in an expanded configuration. The frame porosity may vary in different portions of a sidewall. Thus, for a given selected portion of a frame sidewall, the frame porosity is the total area of a selected portion of a sidewall minus the area of the struts defining the selected portion of the sidewall, divided by the total area of the selected portion of the sidewall when the device is in an expanded configuration.
Cover—A film, membrane, or coating connecting two or more struts of a frame and extending over some or all of the open area of the sidewall defined by the struts of the frame. In most cases, a cover will comprise an outer layer of material extending over the top of the struts and between the struts.
Cover Porosity—The fractional or equivalent percent open pore area of a selected portion of a cover when the device is in an expanded configuration. Cover porosity may vary in different portions of a sidewall that is covered by the membrane. The term “cover porosity” excludes those portions of the cover which are blocked by wires of the frame.
Flow Diverter Porosity—The fractional or equivalent percent open area of a selected portion the side wall of a diverter including both the cover and the frame. A corresponding parameter is flow diverter surface coverage (defined below) which expresses the fractional or equivalent percent blocking portions of the sidewall. Flow diverter surface coverage is 1 minus flow diverter porosity (or 100 minus flow diverter porosity when expressed as percentages).
Pore Size—The size of a pore may be defined by a pore's areal equivalent diameter, which is defined as the diameter of a circle with the same area as the pore. Accordingly, a pore areal equivalent diameter for a given pore may be calculated as the square root of (4A/pi) where A is the measured pore area. Pore size measurements may be made by microscope imaging of the cover or a portion thereof where open portions of the cover appear as light or dark pixels and solid blocked portions of the cover appear as the opposite dark or light pixels. Image processing can be used to identify discrete contiguous open regions which correspond to pores. The area of such an identified open region can be characterized by a pixel count corresponding to the region which can then be converted to an area measurement using the magnification and pixel resolution of the imaging system. The area measurement can be converted to an areal equivalent diameter with the formula above to obtain the pore size for the pore.
Pore Aspect Ratio—The ratio of long side to short side of a rectangle circumscribing a pore.
Cover Porosity Distribution—The cover porosity in a selected region of a cover may be distributed among groups of pores having particular pore characteristics, usually size characteristics. For example, a cover with 30% cover porosity may have a certain fraction of that porosity contributed by pores with a defined size range. The cover porosity distribution refers to a characterization of the amount of total porosity contributed by pores with a defined set of one or more properties.
Cover Permeability—Cover permeability is a qualitative or quantitative measure of the ability of different substances to pass through the cover when the device is in normal use in a vessel. Cover permeability is affected by several different aspects of a cover, including cover porosity and porosity distribution with respect to different size components of blood and/or particles in blood as well as the chemical properties of the cover material with respect to the chemical properties of different components of blood and/or particles in blood. Cover permeability to various blood components can also be affected by local flow and pressure conditions at the site of implantation in a vessel. The characteristics of the fluid flows encountered during use of the devices described herein where porosity and permeability are relevant device properties will be apparent from the context and typically involve blood flow through the otherwise unobstructed ostia of intracranial blood vessels and/or necks of intracranial aneurisms over or across which the device is to be applied.
Flow Diverter Surface Coverage—The fractional or equivalent percentage closed area of a selected portion of the outer surface of a flow diverter in an expanded configuration, where the closed area includes both the cover area outside the pores and frame area where a frame wire blocks pores.
Electrospinning—A technique for depositing a layer of fibers onto a target surface that involves expelling a jet of polymer solution in a reservoir from an orifice to the target surface under the influence of an electric field. By moving the orifice and/or the target surface during the electrospinning process, polymer fibers and fibrous polymer layers and mats having a variety of characteristics can be created. A fiber or fibrous polymer layer or mat so deposited is referred to herein as “electrospun.” A variety of electrospinning techniques and materials suitable for electrospinning are well known. Examples are described in paragraphs [0061] to [0077] of U.S. Patent Publication 2018/0161185 to Kresslein et al., which paragraphs are incorporated herein by reference.

The present disclosure relates to intravascular flow diverters and related methods of using and/or manufacturing the same. Several example embodiments of such intravascular flow diverters, and related methods, will now be described in connection with one or more figures.

Endovascular repair of fusiform aneurysms or bifurcation aneurysms is difficult due to complex flow geometries, side branching blood vessels at the bifurcation and the potential for perforating blood vessels that it is desirable to preserve. Accordingly, a need exists for improved intravascular flow diverters and related methods of using and/or manufacturing the same.

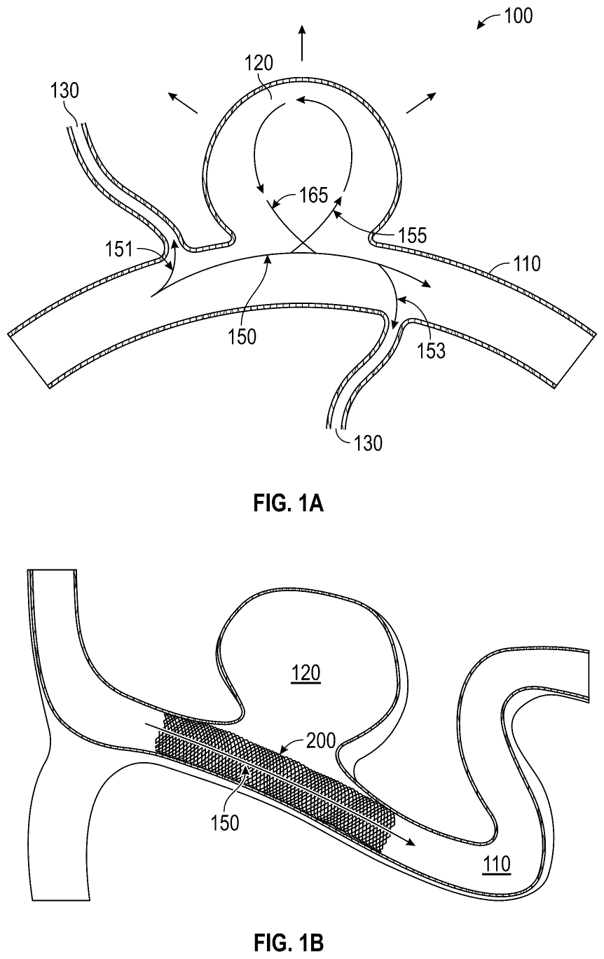

FIG. 1A illustrates a portion of a blood vessel 100. Artery 100 is illustrated as including one or more side branching (also referred to as perforator) blood vessels 130 and an aneurysm 120, shown bulging beyond adjacent portions of blood vessel 100. FIG. 1A illustrates blood flow through the vessel 110 in the region around the aneurysm 120. In many cases, especially for wider necked aneurysms, some of the vessel blood flow 150 past the aneurysm neck is diverted into the aneurysm as aneurysm inflow 155, where it may circulate backwards and then re-enter the vessel flow 150 as aneurysm outflow 165. This intrasaccular circulation pushes outward on the aneurysm wall, causing expansion and possibly rupture. Endovascular repair of fusiform aneurysms or bifurcation aneurysms is difficult due to complex flow geometries, side branching blood vessels at the bifurcation, and the potential for perforating blood vessels with blood flows 151, 153 that are desirable to preserve.

Treatments for such types of aneurysms 120 can involve the use of flow diverters to bypass the diseased area. One contemporary treatment involves the use of Medtronic's Pipeline flow diverter, having 64×20 to 30 micrometer diameter wires in a braid, configured to bridge over aneurysm 120. This is illustrated generally in FIG. 1B. Resistance to blood flow through the sidewall of such a pipeline flow diverter 270 is provided by this wire braiding. However, this wire braiding which constructed from a large number of cobalt chromium wires, can result in these pipeline flow diverters being relatively stiff and difficult to deploy. Additionally, to completely occlude flow to some aneurysms 120, two or more such flow diverters may need be deployed concentrically within one another to achieve the desired inflow stasis to aneurysm 120. This makes for a costly and complicated procedure.

For these reasons and others, a need exists for improved flow diverters for aneurysm treatment and related methods of using and/or manufacturing the same. Accordingly, in some embodiments described herein, an intravascular flow diverter for treating an aneurysm of a blood vessel is provided. The diverter includes a frame including a plurality of wires coiled to form an expandable structure. The diverter includes a cover disposed on at least some of the outer surface of the frame. The cover includes a first portion configured to be disposed directly against a neck/ostium of the aneurysm. The cover includes at least one second portion configured to be disposed adjacent to and not directly over the aneurysm. The characteristics of the cover can be the same or different in different areas of the flow diverter. Common locations for intracranial aneurysms include the communicating arteries, the internal carotid arteries, and the middle cerebral artery. The devices described herein can, for example, be used in these arteries.

In some other embodiments, a method for utilizing an intravascular flow diverter to treat an aneurysm of a blood vessel is provided. The method includes disposing the intravascular flow diverter within a microcatheter. The method includes threading the microcatheter through the blood vessel to a location of the aneurysm. The method includes removing the intravascular flow diverter from a distal end of the microcatheter such that a frame of the intravascular flow diverter, including a plurality of wires coiled to form an expandable structure, expands sufficiently within the blood vessel that a first portion of a cover disposed over at least a first portion of the frame is disposed directly against a neck/ostium of the aneurysm, and that at least a second portion of the cover is disposed adjacent to and not directly over the aneurysm.

In some other embodiments, a method of manufacturing an intravascular flow diverter configured for treating an aneurysm of a blood vessel is provided. The method includes coiling a plurality of wires to form an expandable frame. The method includes depositing or otherwise affixing a cover over the frame.

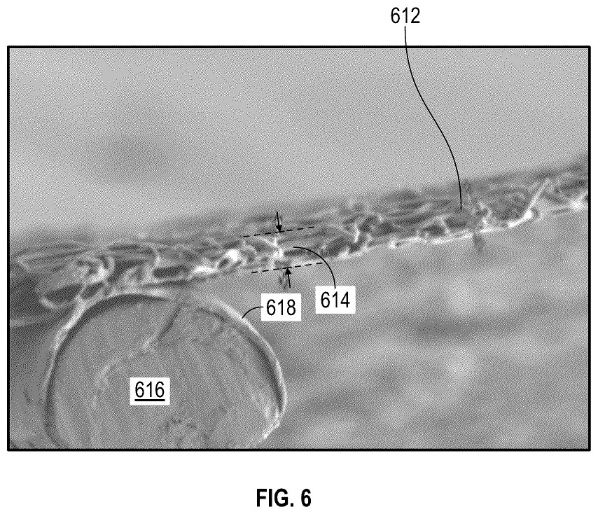

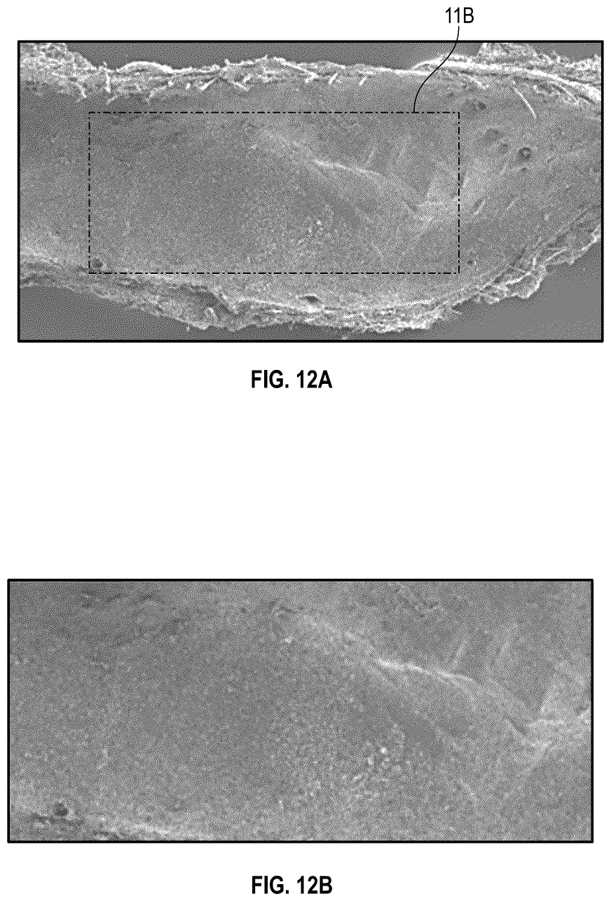

As described further below, intravascular flow diverters as described herein may include a frame which may comprise a self-expanding and/or balloon expandable set of wires or struts 420 (which may be constructed from known techniques including, but not limited to, braiding and/or laser cutting) and coated with a cover 410 comprising one or more portions 310, 320a and 320b having one or more desired pore characteristics, which may be the same, substantially the same, or substantially different in the three regions 310, 320a, 320b. Particular advantageous embodiments of intravascular flow diverters 300 will be described in more detail in connection with at least FIGS. 6-12.

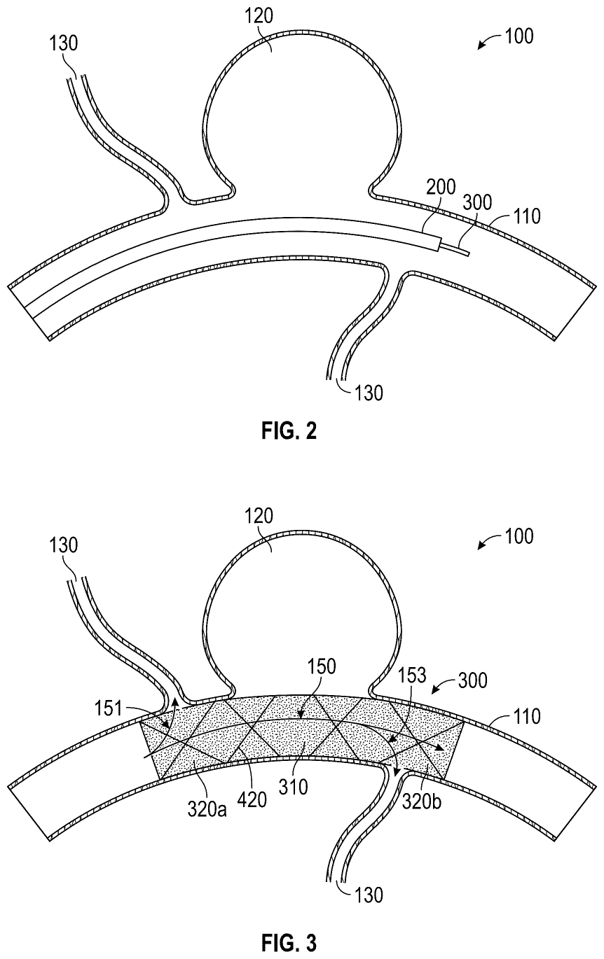

As illustrated in FIG. 2, such a flow diverter 300, comprising such a collapsible frame and cover, may be disposed within a microcatheter 200 in a collapsed configuration and the microcatheter 200 threaded through blood vessel 100 until flow diverter 300 is disposed adjacent aneurysm 120 with a goal of substantially restricting blood flow into aneurysm 120 and, thereby, reducing stress induced on a wall of aneurysm 120.

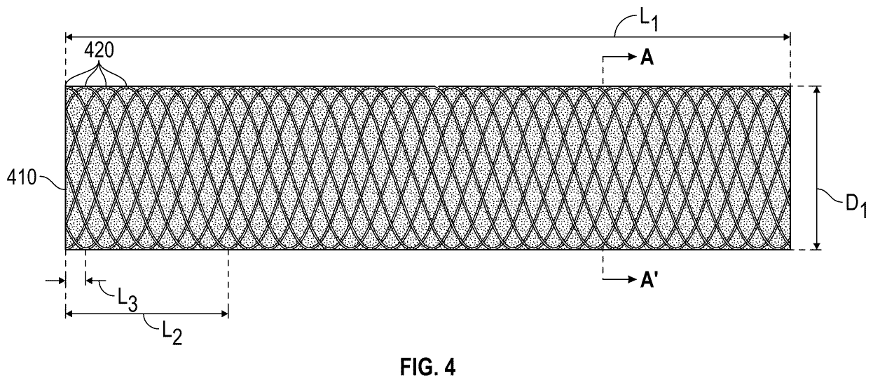

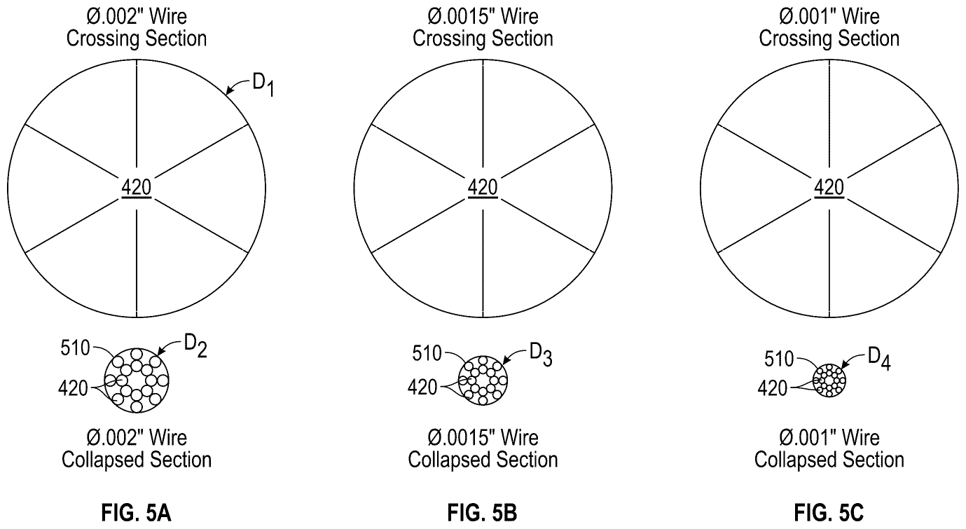

Once properly disposed adjacent aneurysm 120 within blood vessel 100, the covered frame of flow diverter 300 may be allowed to self-expand under bias from the plurality of wires or struts 420 (see, e.g., FIGS. 4-5C) and/or may be manually expanded utilizing an expansion balloon disposed within at least a portion of the collapsed frame (not shown in the figures).

Referring now to FIG. 3, intravascular flow diverter 300 may comprise a first portion 310 configured to be disposed directly over a neck/ostium of aneurysm 120. Intravascular flow diverter 300 may further comprise one or more second portions 320a, 320b adjacent to first portion 310 and configured to be disposed along the vessel 110 adjacent to the aneurysm 120. There may be side branching perforator vessels 130 that receive blood flow from the main vessel 110 in these regions adjacent to the neck/ostium of the aneurysm. It is generally advantageous to retain normal or near normal blood flow into the side branches 130 of blood vessel 110 as compared to the significantly reduced or even substantially eliminated blood flow 155, 165 (FIG. 1A) into and out of aneurysm 120. Embodiments of the invention may have substantially the same pore characteristics over the whole cover or substantially the whole cover. In some cases, this is a preferred configuration for reasons of manufacturability as well as ease of implantation.

As illustrated in FIG. 4, flow diverter 300 comprises a plurality of wires 420 braided or otherwise wound around and/or adjacent to one another. Each wire may be 25 to 125 microns in diameter. In some embodiments, each of wires 420 may be coiled into a collapsible and expandable, substantially helical shape or structure configured to have a predetermined length L₁ (e.g., 10 to 40 mm, although any other suitable length is also contemplated) and a predetermined maximum diameter D₁ in an expanded configuration (e.g., 1 to 6 mm, although any other suitable diameter is also contemplated) when fully expanded. In some such embodiments, each wire 420 is offset from adjacent wires 420 by a predetermined spacing L₃ (e.g. 0.1 to 0.5 mm). In some embodiments, each of wires 420 may have a predetermined pitch L₂ (i.e., each loop or winding of a particular one of wires 420 extends predetermined length L₂ (e.g., 1 to 5 mm mm, although any other suitable length is also contemplated) along a length of extension of flow diverter 300. In some such embodiments, the spacing L₃ may be determined as the result of dividing pitch L₂ of wires 420 by a number of those wires that are wound in a same direction. For example, in some embodiments, a subset (e.g., 8 of the plurality of wires 420) of the plurality of wires 420 are wound in a clockwise direction, while another subset of the plurality of wires 420 (e.g., another 8 of the plurality of wires 420) are wound in a counterclockwise direction. Accordingly, where subsets of wires 420 are wound in opposite directions from one another, one subset of wires 420 will overlap the other subset of wires 420 at multiple points along the predetermined length L₁.

In some embodiments, the frame is configured to elongate while collapsing to, thereby, minimize a collapsed diameter of the frame. The above and below-described geometries of wires 420 advantageously provide for easy, unobstructed expansion of flow diverter 300 in vivo. In some embodiments, wires 420 comprise super-elastic nitinol. In some other embodiments, a cobalt chromium or platinum may be used. In yet other embodiments, a nitinol shape memory alloy may be used. Different wires of the same frame may be made of different materials. However, the present disclosure is not limited to particular materials and wires 420 may comprise any suitably flexible material.

Each of wires 420 may be further coated with a polymer 510 which may be a dip coating configured to substantially reduce or minimize metal exposure to the blood. The dip coating can also bind the wires together at the crossing points. In some embodiments, polymer 510 comprises an elastomeric polymer.

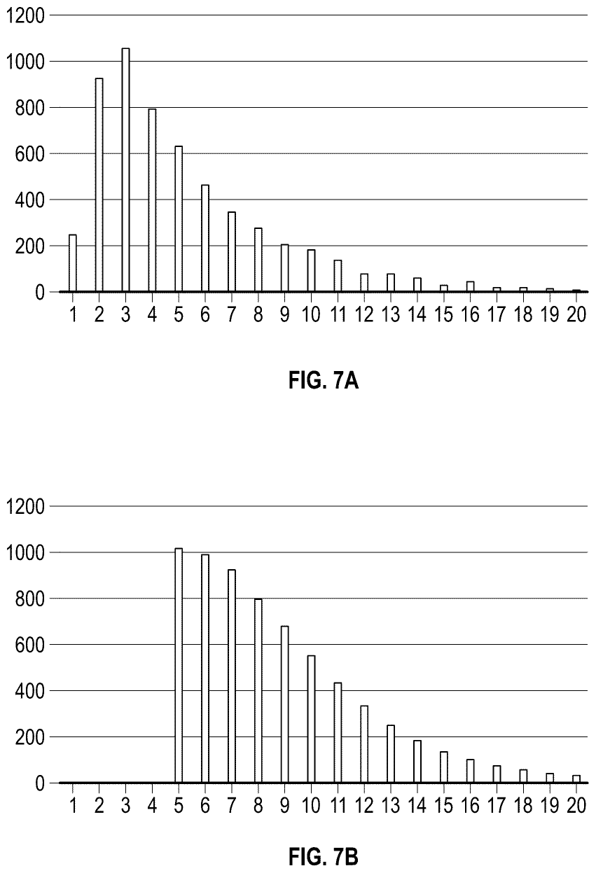

As further illustrated in FIG. 4, flow diverter 300 comprises a cover 410 disposed on at least a portion of the outer surface, which may be an entire outer surface, e.g., substantially around an entire perimeter, of flow diverter 300 and along the entire length of extension L₁. It can be advantageous in some embodiments though to have the cover be applied only over a central region of the frame, with bare frame struts extending out from one or both sides of the covered portion. For example, in some embodiments, membrane 410 has a substantially cylindrical form when the entirety of flow diverter 300 is disposed parallel to the length of extension L₁, as shown in FIG. 4. Cover 410 may provide support for the underlying expanded frame of wires 420 as well as a controlled porosity layer that, advantageously, has a relatively thin construction, for example having a thickness 614 (FIG. 7) as a result of an electrospinning process utilized to form cover 410. The above-mentioned dip coating when provided can provide a good bond between an electrospun membrane and the frame.

Cover 410 allows a reduction in a number of wires 420 (e.g., 6, 8, 12 or 16 of wires 420) needed for construction and effective operation, for example compared to the Medtronic pipeline flow diverter (e.g., having and requiring 48 or 64 wires). For example, whereas such Medtronic Pipeline flow diverters rely on the density and close proximity of the many (e.g., 64) individual wires to provide sufficient support to the blood vessel wall and to also provide sufficiently low porosity to inhibit blood flow to the aneurysm, at least some of the requisite support to the wall of blood vessel 100 and the majority of the requisite low porosity/high coverage of flow diverter 300 are provided by cover membrane 410.

Cover 410 may provide support for the underlying expanded frame of struts 420 while also possessing a relatively thin construction. A cover membrane 410 providing such support allows a reduction in a number of wires 420 needed for construction and effective operation, compared conventional devices not comprising such a membrane. The cover membrane allows an increase in frame porosity. In some embodiments, the frame porosity over a majority, substantially all, or all of its overall length is greater than 0.90 (which it will be appreciated can be expressed as the percentage 90%), preferably greater than 0.93(93 %), more preferably greater than 0.95(95 %).

This reduction in strut area (increase in frame porosity) also advantageously reduces device mass per unit length, delivery profile, longitudinal stiffness of, and radial force exerted by, device 300 during navigation to aneurysm 120 in the delivery system. All of these improvements separately and collectively allow for easier tracking into the vasculature and improved delivery and deployment of device(s) 300. This is especially true, and advantageous, for applications to smaller and/or tortious blood vessels, such as those of the brain, where the ratio of collapsed-to-appropriately deployed radii of device(s) may be much smaller than for applications to larger blood vessels, such as the aorta.

This reduction in wire number also advantageously reduces a delivery profile and longitudinal stiffness of, as well as a radial force exerted by, diverter 300 during navigation to aneurysm 120 in the delivery system. All of these improvements separately and collectively allow for easier tracking into the vasculature and improved delivery and deployment of diverter 300. This is especially true, and advantageous, for applications to smaller and/or tortious blood vessels, such as those of the brain, where the ratio of collapsed-to-appropriately deployed radii of diverter 300 may be much smaller than for applications to larger blood vessels, such as the aorta.

Moreover, the reduced number of wires 420 also provides more uniform porosity across the neck of a curve of diverter 300 at least because the smaller number of wires crowd to a comparatively lesser degree on the inside of such curves. And, because porosity along a length of diverter 300 is largely controlled by the porosity and/or permeability of membrane 410, the porosity and/or permeability of membrane 410 can be tailored to restrict blood flow through areas of membrane 410 immediately over or against the diseased blood vessel wall of aneurysm 120 (see, e.g., the portion of membrane 410 shown in FIG. 4 forming first portion 310 shown in FIG. 3) to, thereby, allow clots to form, organize and scar over to produce an effective long-term treatment, while simultaneously allowing sufficient blood flow to or through areas of membrane 410 adjacent to but not over or directly against the diseased blood vessel wall of aneurysm 120 (see, e.g., the portions of membrane 410 forming the lateral second portion(s) 320 in FIG. 3) to maintain patency of side branches 130 of blood vessel 100.

Although it can be advantageous to have the same or substantially the same cover characteristics such as thickness and pore characteristics over the whole cover, in some embodiments, a density and/or a porosity of membrane 410 may be tuned to have different or variable values at different locations and, thereby, provide for relatively high porosity of second portion(s) 320 (for positioning adjacent side branches 130) and for the relatively low porosity first portion 310 (for positioning at aneurysm 120, where isolation from blood flow and clotting external to the stent should be promoted). In such embodiments, areas of blood vessel 100 having perforating vessels or side branches 130 that require maintained blood flow advantageously remain viable by virtue of the increased blood flow through second portion(s) 320 of membrane 410. The above-described advantages may also directly reduce a need for physicians to use multiple flow diverters to achieve stasis of blood flow into aneurysm 120.

Additionally, flow diverter 300 may have an improved ability for use with coils, or intrasaccular flow diverters. For example, in the event of inadvertent rupture of an aneurysm while using flow diverter 300, in combination with such coils or intra-saccular flow diverters, flow diverter 300 provides additional hemostasis via membrane 410 compared to traditional dense wire braid stents.

It is also contemplated that wires 420 may have one of a variety of different diameters, according to requirements of a desired application. FIGS. 5A-5C illustrates cross-sections of first through third example embodiments of intravascular flow diverter 300, for example, as viewed along a cutline A-A′ shown in FIG. 4. Each of the embodiments of FIGS. 5A-5C may be substantially similar to one another, except each utilizes wire 420 having a different diameter and, therefore, each illustrated embodiment of diverter 300 also comprises a respective minimum collapsed diameter.

As illustrated in FIG. 5A, wires 420 may have an outside diameter of approximately 0.002 inches, and diverter 300 may, accordingly, be configured to have a minimum contracted diameter D₂ (e.g., 0.011 inches) and the maximum expanded diameter D₁.

As illustrated in FIG. 5B, wires 420 may have an outside diameter of approximately 0.0015 inches and diverter 300 may, accordingly, be configured to have a minimum contracted diameter D₃ (e.g., 0.008 inches) and the maximum expanded diameter D₁.

As illustrated in FIG. 5C, wires 420 may have an outside diameter of approximately 0.001 inches, and diverter 300 may, accordingly, be configured to have a minimum contracted diameter D₄ (e.g., 0.006 inches) and the maximum expanded diameter D₁.

The example orientation of wires 420 in the collapsed state illustrated in each of FIGS. 5A-5C comprises an innermost subset of 8 threads and an outermost subset of 8 threads. Each of the innermost threads is disposed in direct contact with each of two adjacent threads of the innermost subset and one thread of the outermost subset. A center of each of the outermost threads is radially in-line with both a center of the frame's cross section and a center of the corresponding inner thread with which the outermost thread is in direct contact.

In some embodiments, coiling wires 420 comprises winding each of wires 420 into a substantially helical shape. In some embodiments, wherein each of wires 420 are wound into the substantially helical shape such that each of a plurality of helical loops has a predetermined pitch L₂. In some embodiments, a first subset of wires 420 are wound in a clockwise direction and a second subset of wires 420 are wound in a counterclockwise direction. In some embodiments, each of wires 420 is offset from at least one adjacent wire 420 by a predetermined spacing L₃. In some embodiments, the plurality of wires 420 is one of 6 wires, 8 wires, 12 wires and 16 wires. In some embodiments, wires 420 are configured to self-expand under a self-bias. In some embodiments, wires 420 are configured to expand under influence of a balloon configured to be expanded from within the substantially helical shape. In some embodiments, intravascular flow diverter 300 is configured to be disposed, in a collapsed form, within microcatheter 200 that is configured to be threaded through blood vessel 100 to a location of aneurysm 120 (see, e.g., FIG. 2). In some embodiments, at least one of the at least one second portions 320 is configured to be disposed directly over side branch 130 of blood vessel 100. In some embodiments, the first porosity is sufficiently low that, when intravascular flow diverter 300 is properly disposed within blood vessel 100, first portion 310 of membrane 410 is configured to allow substantially no blood flow therethrough to, thereby, allow a clot to form and ultimately scar over at aneurysm 120. In some embodiments, the second porosity is sufficiently high that, when intravascular flow diverter 300 is properly disposed within blood vessel 100, at least one of the at least one second portions 320 of membrane 410 are configured to allow substantial blood flow therethrough and directly into side branch 130 of blood vessel 100. In some embodiments, each of wires 420 is provided to have a diameter of one of approximately 0.002 inches, approximately 0.015 inches, and 0.001 inches (see, e.g., FIGS. 5A-5C). In some embodiments, intravascular flow diverter 300 is configured to have a minimum outside diameter D₂, D₃, D₄ of one of approximately 0.011 inches, approximately 0.008 inches, and approximately 0.006 inches when the expandable structure of the stent is fully collapsed (see, e.g., FIGS. 5A-5C). In some embodiments, intravascular flow diverter 300 is configured to have a maximum outside diameter D₁ of approximately 0.18 inches when the expandable structure of the stent is fully expanded (see, e.g., FIGS. 5A-5C). In some embodiments, a substantial majority of an aggregate porosity of intravascular flow diverter 300, at the first and second portions 310, 320 of membrane 410, is derived from the first and second porosities of membrane 410 at the respective first and second portions 310, 320. In some embodiments, each of wires 420 comprises at least one of a super-elastic nitinol, cobalt chromium, and a nitinol shape memory alloy.

FIG. 6 shows a magnified image of a cover 612 attached to a wire 616 of a frame. Also shown is a polymer coating 618 on the wire, which may be polyurethane, which helps the cover stick to the wires and reduced friction during deployment. The cover 612 has a thickness 614 which is preferably less than 10 microns or more preferably less than 7 microns. As the individual electrospun filaments themselves may typically have a diameter of about 1-2 microns, cover thickness normally won't be below 2 microns. In the embodiment shown in FIG. 6, the thickness of the cover is between 4.5 and 5.5 microns. The thickness of the cover can vary in different portions of the flow diverter either intentionally (e.g. the cover may be made thicker in the central area 310 that extends over the neck of the aneurysm) or due to variations occurring naturally during manufacturing. It is generally advantageous to maintain a relatively consistent thickness (e.g. within 1 micron of an average thickness) over at least most of the flow diverter, with the above thickness dimensions/ranges/consistencies preferably being maintained over at least 70% of the cover area, preferably over the entire cover area. Advantageous pore characteristics for the cover are discussed in further detail below, and it is also preferable for the cover to have relatively consistent pore characteristics over at least 70% of the cover area, preferably over the entire cover area. A cover with relatively consistent construction in terms of thickness and pore size characteristics has advantages in ease of manufacturing. In addition, a flow diverter is easier to install if there is flexibility for the surgeon in angular orientation and precise positioning of the device because it is not necessary to accurately orient a particular portion of the cover with a particular anatomical feature of the vasculature. When discussed herein, if a characteristic of a flow diverter cover is described, unless otherwise stated it is intended that the characteristic applies to at least 70% of the cover area, but not necessarily 100% of the cover area. However, in most embodiments, the manufacturing intention is to provide relatively consistent cover parameters over the whole cover of the device.

There are three fundamental biological functional parameters that flow diverter performance can be characterized by for flow diverters intended for long term implantation across the ostium of an aneurysm in a blood vessel. These are (1) degree of blocking of blood flow into and out of the sac of the aneurysm to allow embolization of the aneurysm, (2) degree of maintaining blood flow into adjacent perforating vessels near the aneurysm, and (3) incorporation of the flow diverter into an endothelial layer forming part of the vessel wall. The inventors have developed flow diverter covers with pore characteristics that meet these goals with a cover 410 that can be substantially uniform over its functional length through all three regions 310, 320a and 320b illustrated in FIG. 3. This is accomplished in some embodiments of the invention by a cover 410 that has porosity distributed over an appropriate variety of size ranges.

FIGS. 7A and 7B are pore size histograms for two covers that experiments have shown to have good biological performance characteristics. In these Figures, the y-axis is pore count, and the x-axis are 1 micron width pore size bins, where pore size is as defined above, the areal equivalent pore diameter. The 1 micron width bins include pores with areal equivalent diameters less than but within 1 micron of the bin label. For example, the bin labeled 6 (the “6 micron bin”) shows the number of pores less than 6 micron areal equivalent pore diameter and greater than or equal to 5 micron areal equivalent pore diameter.

Contrary to expectations, initial experiments with larger pore sizes, e.g. 30 micron, 50 micron, and the like did not perform as desired in terms of flow blockage into the aneurysm, and further experiments were performed with covers having a preponderance of much smaller pore sizes. The smaller pore sizes improved aneurysm inflow blocking, and it was further surprisingly found that the change to smaller pore sizes did not significantly degrade the cover's ability to maintain patency of adjacent perforating vessels. The inventors have found that for covers with good performance as flow diverters the median areal equivalent pore diameter should be surprisingly small, such as between 3 and 9 microns. In addition, it was found preferable that there be very few pores (e.g. less than 5% of the pores by pore count or less than 2% of the pores by pore count) having areal equivalent diameters greater than 20 microns. Additionally, in many advantageous embodiments, 5% to 20% of the pores have an areal equivalent diameter of greater than 12 microns. Without being bound by any particular theory of operation, it appears that having a sufficient fraction of pores greater than typical red blood cell diameter limits pressure drop across the openings of perforating branch vessels from the primary vessel. Experiments in rabbits have demonstrated this functionality in small perforating arteries feeding the spine from the descending aorta in rabbits and in high flow arteries such as the vertebral artery in rabbits. The larger pores in the 12 to 20 micron range coupled with a thin cover layer produce relatively small pressure drop across it at the perforator entry points and can provide blood flow into the side branching vessels while at the same time the preponderance of small pores (e.g. less than 9 micron pore size), small pore size median, and substantial absence of pores greater than 20 microns still results in effective blockage of blood flow into and out of the aneurysm itself.

As can also be seen in FIGS. 7A and 7B, it has been found advantageous for the pore count (per micron width size bin) to generally increase over decreasing micron width bins from 20 microns to at or below the approximate diameter of red blood cells at around the 8 or 9 micron bins. Thus, the pore counts for the 10 and 11 micron bins are greater than the 19 and 20 micron bins and the pore counts for the 7 and 8 micron bins are greater than the pore counts for the 10 and 11 micron bins. Restricting the pore sizes to (1) almost all pores below 20 microns in size, (2) a significant number of pores with sizes greater than 12 microns, and (3) a significant number of pores with sizes less than 8, less than 5, or even less than 3 microns (generating a low median pore size) provides a good balance of blocking blood flow into the aneurysm and maintaining blood flow into adjacent perforating vessels.

In some embodiments, the median areal equivalent diameter of the pores is between 3 and 5 microns. For these embodiments, 20% to 45% of the pores by pore count may have areal equivalent diameters of less than 3 microns. In some embodiments, the median areal equivalent diameter is between 6 and 9 microns. For these embodiments, less than 5% of the pores by pore count may have areal equivalent diameters of less than 3 microns In the 6 to 9 micron median pore size embodiments, about 50% of the pores have areal equivalent diameters less than the typical diameter of red blood cells and about 50% of the pores have areal equivalent diameters greater than the typical diameter of red blood cells. Without being bound by any particular theory of operation, it is believed that the above described pore size distributions, especially but not exclusively those with an approximately equal number of pores smaller than red blood cells and pores larger than red blood cells produces a desired balance between blocking blood flow into the sac of the aneurysm and allowing blood flow into perforator vessels adjacent to the aneurysm. Furthermore, limiting the number of larger pores greater than 20 micron areal equivalent diameter prevents such larger pores from upsetting this balance.

Regarding the ability of the device to be successfully incorporated into an endothelial layer of the vessel wall, the inventors have found that the thin cover (e.g. less than 10 micron thickness, or in some advantageous embodiments less than 7 micron thickness) and pores almost all of which are less than 20 micron areal equivalent diameter fosters this incorporation. However, it is believed that providing a significant number of small pores with less than 5 micron areal equivalent diameter further enhances endothelium incorporation. In some of these advantageous embodiments, 10% to 70% of the pores by pore count over at least 70% of the cover area have an areal equivalent diameter of less than 5 microns. Again, without being bound by any theory of operation, it is believed that providing a relatively large number of small pores less than 5 microns promote endothelialization by allowing endothelial cells to overlay multiple small pores in a substantially flat orientation. In addition, the thinness of the membrane means relatively low tortuosity of the cover from the surface of the vessel to endothelial cells on the inner surface of the cover allowing relatively free flow of factors and nutrients that contribute to forming a healthy endothelial layer. The thin cover described herein thus becomes not a substitute for the extracellular matrix in which an endothelium forms, but is a substrate that can be permeated by it, which then naturally fosters endothelium creation over the cover following implantation. In contrast, thicker covers and large pores tend to produce gaps and voids in the post implantation endothelium. In some embodiments, these effects are enhanced by providing a significant number of pores having areal equivalent diameters of less than 3 microns. In one embodiment that is illustrated in FIGS. 7A, 20% to 45% of the pores have areal equivalent diameters of less than 3 microns and the median areal equivalent diameter is less than 5 microns, such as between 3 and 5 microns.

An advantageous feature of some embodiments is an average pore aspect ratio of greater than 1, such as between 1.1 and 1.4. For smaller pores, this may further enable endothelialization whereas for larger pores, it may further enable maintenance of blood flow into perforating branch vessels because red blood cells can potentially pass through edgewise even for pores of smaller areal equivalent diameters.

Another aspect that can be significant to the performance of the flow diverter is the flow diverter surface coverage (which as set forth above may correspond to 1 minus the flow diverter porosity). A balance between perforator flow and aneurysm blocking is important for this parameter as well. In some embodiments, the flow diverter surface coverage is between 0.45(45 %) and 0.85(85 %). In some embodiments, the flow diverter surface coverage is between 0.60(60 %) and 0.70(70 %). These surface coverages in conjunction with the pore size distributions set forth above provide the appropriate permeability balance to adequately block blood flow into an aneurysm, adequately allow blood flow into adjacent perforating vessels, and foster incorporation into a post implantation formed endothelium on the vessel wall.

Another advantage of cover 410 being applied to the frame of wires 420 is that the flow control achieved by the cover 410 can be easily manufactured with a variety of characteristics, and flow diverters with different characteristics can be provided as part of surgical kits for aneurysm treatment. For example, surgical kits can be provided with a first plurality of flow diverters having a first range of pore characteristics and a second plurality of flow diverters having a second range of pore characteristics. The first and second pluralities can be segregated or grouped to be distinguishable in the kit so that a surgeon can select the pore characteristics especially suitable for the procedure being performed. As the flow diverters will generally be provided pre-loaded in a collapsed state inside microcatheters for deployment, they can be segregated by marking, separate packaging, or any other means. As an example of such a kit, a first set of flow diverters may have a median pore size of 4 microns and a second set of flow diverters may have a median pore size of 8 microns. When a surgeon is using the kit, the surgeon may choose to implant one of the smaller median pore size flow diverters when there are no significant perforating vessels adjacent the aneurysm to be treated. In such a case, the surgeon may select the smaller median pore size to optimize aneurysm blocking and endothelial incorporation. On the other hand, in the presence of perforators near the aneurysm to be treated the surgeon may select one of the larger median pore size flow diverters to better insure patency of the perforators to be maintained. It will be appreciated that different groups with different pore size ranges can be provided, and three or more sets of flow diverters with different characteristics could be provided in a surgical kit. As another distinguishing feature, flow diverter surface coverage could be another characteristic provided in different groups or sets of flow diverters in a kit.

Providing different sets of flow diverters with different pore characteristics may not significantly increase the number of flow diverters provided in such surgical kits as the use of covers to provide flow control (as opposed to wire frames alone) will advantageously allow fewer sizes of diverter 300 to be made available and/or used compared to existing flow diverters, all of which have fairly narrow ranges of vessel diameters for which each size is effective at segregating blood flow. For example, traditional wire braids are generally provided in ¼ mm increments while, at least in some embodiments, flow diverter 300 may be made available in expanded configuration sizes with much larger increments, e.g., 1.0 to 1.5 mm, thereby allowing 4-6 fewer sizes of diverters 300 than compared to traditional wire braid stents. In the case of two or more sets of flow diverters with different median pore size, each set can be provided in size increments of, for example, 0.5 mm, 1.0 mm, or size increments greater 1.0 mm.

### Example 1

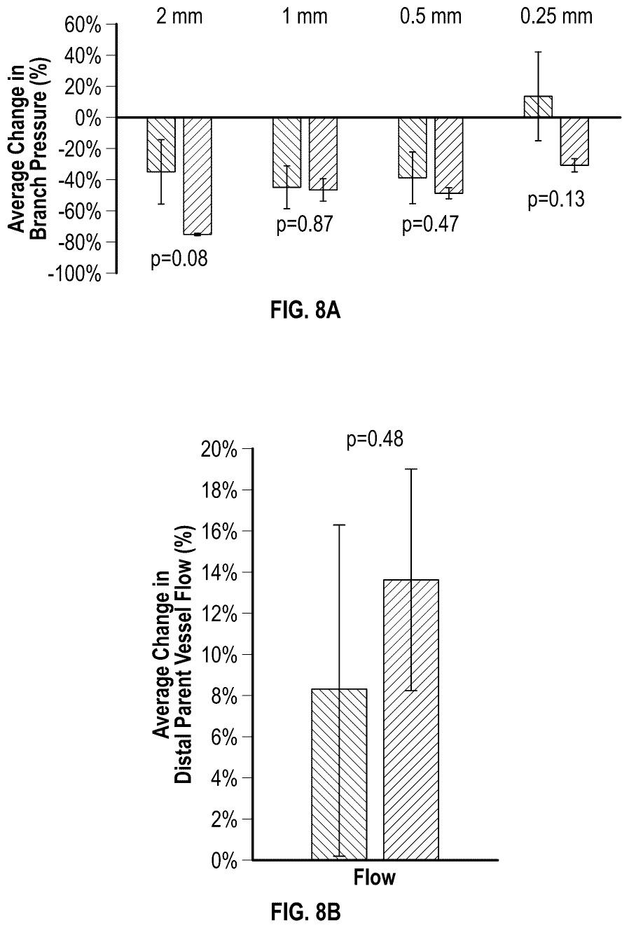

A physical model of a vascular structure with a parent vessel and a branching vessel was created and performance of embodiments of the invention characterized by pore distributions similar to that shown in FIG. 7B were compared to performance of Medtronic Pipeline flow diverters in the model. FIG. 8A shows pressure changes at four different locations in the branching vessel after installation of the diverters. The left bars show the average results of three invention embodiments and the right bars show the average results of two Medtronic Pipeline flow diverters. As shown in FIG. 8A, the pressure drop across the diverter wall from the parent vessel to the perforating vessel is lower for the invention embodiments than for the Medtronic Pipeline.

FIG. 8B shows the average change in parent vessel flow for the same three invention embodiments (left bar) and the same two Medtronic Pipeline flow diverters. As shown in FIG. 8B, parent vessel flow is less disturbed by the invention embodiments than the Medtronic Pipeline diverters.

### Example 2

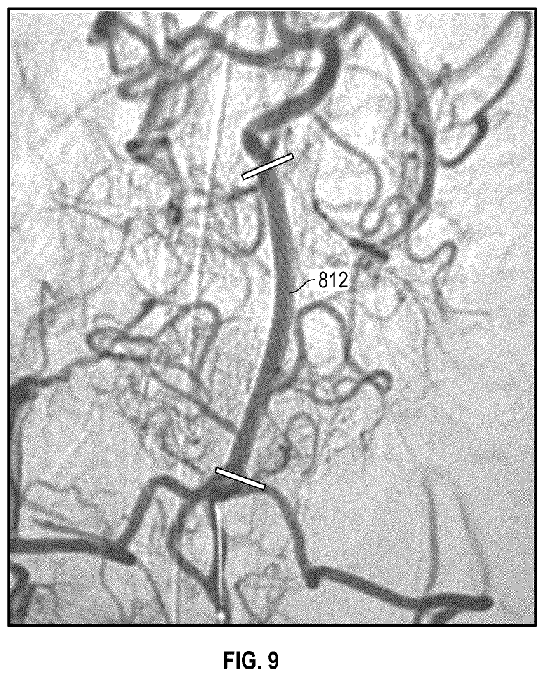

A flow diverter according to the inventive embodiments was implanted in a canine basilar artery. FIG. 9 shows a contrast image of the implanted flow diverter 812. White bars indicate the ends of the implanted flow diverter. Flow into all branching arteries was preserved.

### Example 3

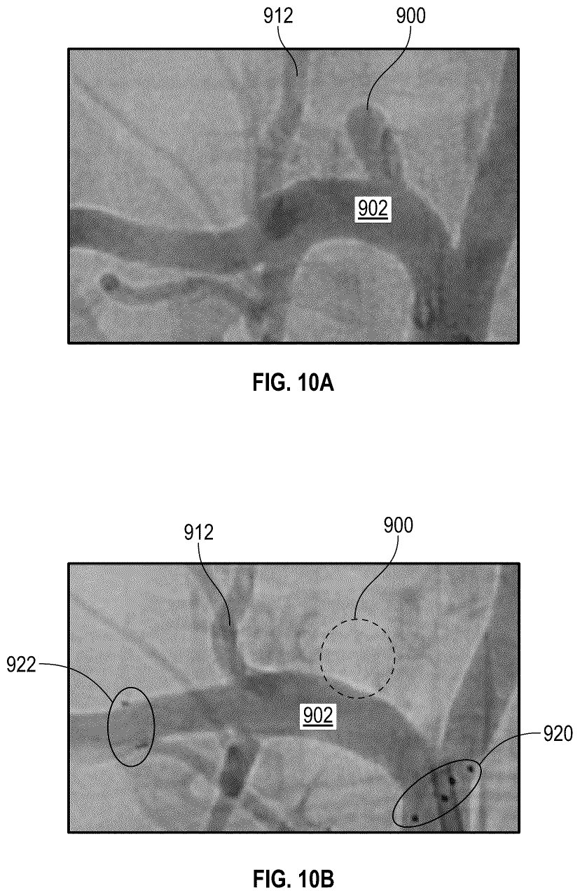

A flow diverter according to embodiments of the invention characterized by pore distributions similar to that shown in FIG. 7A was deployed in the subclavian artery of a rabbit with an elastase induced aneurysm. FIG. 10A illustrates vasculature of the rabbit with contrast showing an aneurysm 900 in the rabbit subclavian artery 902 prior to deployment of the flow diverter. Also shown in FIG. 9A is the perforating vertebral artery 912 branching off of the subclavian artery 902. FIG. 9B illustrates the vasculature of the rabbit under contrast after installation of the flow diverter according to the invention. Radiopaque markers at the proximal side of the diverter 920 and the distal side of the diverter 922 show the extent of the implanted embodiment. As can be seen in FIG. 9B, contrast does not enter the aneurysm sac after the diverter is implanted, indicating sufficient blocking to produce aneurysm embolization. Further shown in FIG. 9B is contrast still entering the branching vertebral artery 912, showing that blood flow continues into the perforating artery.

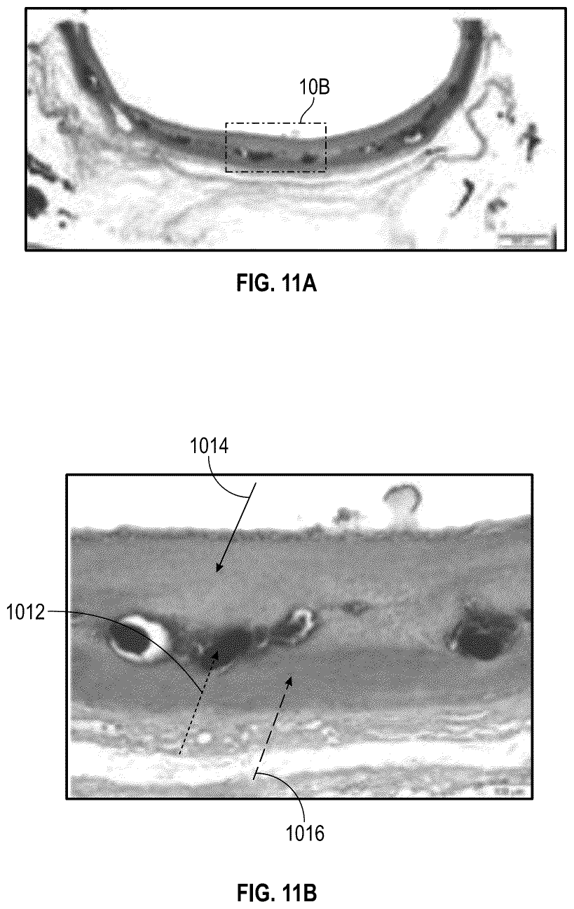

FIG. 10A shows a transversely sectioned vessel half of the rabbit subclavian artery after 30 days of flow diverter implantation. FIG. 10B shows a close-up of the indicated image portion in FIG. 10A. The stent wires (arrow 1012) are covered with a layer of organized neointimal growth (arrow 1014) with intact medial walls (arrow 1016). The cover itself is not visible because the stain used does not distinguish it. FIGS. 11A and 11B show magnified surface images of the rabbit subclavian artery after 30 days of flow diverter implantation. It can be seen in these images that an endothelium has covered over the flow diverter without significant gaps.

### General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

What is claimed is:

1. An intravascular flow diverter for treating an aneurysm of a blood vessel, the intravascular flow diverter comprising:
one or more wires forming a frame, wherein the frame has a collapsed configuration and an expanded configuration, wherein the frame is configured to transition in use from the collapsed configuration to a deployed configuration that substantially conforms to a shape of an inside surface of the blood vessel in the vicinity of the aneurysm;
a cover coupled to and extending over at least a portion of the frame;
wherein the cover comprises pores therethrough over the at least a portion of the frame;
wherein in the expanded configuration a thickness of the cover over at least 70% of the total cover area is less than 7 microns;
wherein in the expanded configuration at least 95% of the pores by pore count over the at least 70% of the total cover area have an areal equivalent pore diameter of less than 20 microns;
wherein in the expanded configuration 5% to 20% of the pores by pore count over the at least 70% of the total cover area have an areal equivalent pore diameter of 12 or more microns; and
wherein in the expanded configuration a median areal equivalent pore diameter of the pores over the at least 70% of the total cover area is between 3 and 9 microns.

2. The intravascular flow diverter of claim 1, wherein in the expanded configuration 20% to 45% of the pores by pore count over the at least 70% of the total cover area have an areal equivalent pore diameter of less than 3 microns.

3. The intravascular flow diverter of claim 2, wherein in the expanded configuration the median areal equivalent pore diameter over the at least 70% of the total cover area is between 3 and 5 microns.

4. The intravascular flow diverter of claim 1, wherein in the expanded configuration less than 5% of the pores by pore count over the at least 70% of the total cover area have an areal equivalent pore diameter of less than 3 microns.

5. The intravascular flow diverter of claim 4, wherein in the expanded configuration the median areal equivalent pore diameter over the at least 70% of the total cover area is between 6 and 9 microns.

6. The intravascular flow diverter of claim 1, wherein the cover extends over at least 70% of the surface area of the frame.

7. The intravascular flow diverter of claim 1, wherein the cover consists essentially of electrospun filaments.

8. The intravascular flow diverter of claim 7, wherein the frame porosity is at least 93%.

9. The intravascular flow diverter of claim 1, wherein in the expanded configuration the flow diverter surface coverage over the at least 70% of the total cover area is between 45% and 85%.

10. The intravascular flow diverter of claim 1, wherein the intravascular flow diverter has an outside diameter of between 1 mm and 6 mm in the expanded configuration.

11. The intravascular flow diverter of claim 1, wherein the one or more wires are configured to self-expand under a bias from the plurality of wires.

12. The intravascular flow diverter of claim 1, wherein each of the plurality of wires has a diameter of between 25 and 125 microns.

13. The intravascular flow diverter of claim 1, wherein the one or more wires comprises eight or fewer wires.

14. The intravascular flow diverter of claim 1, wherein in the expanded configuration an average aspect ratio of the pores over the at least 70% of the total cover area is between 1.1 and 1.4.

15. A method of treating a main vessel sidewall aneurysm, wherein the main vessel has at least one perforating branch vessel adjacent to the sidewall aneurysm, the method comprising:
expanding a flow diverter within the main vessel and against the sidewall of the main vessel to a deployed configuration over a neck of the sidewall aneurysm and over an entry opening of the perforating branch vessel, wherein the flow diverter comprises a cover, wherein the cover comprises pores therethrough, wherein a first portion of the cover is positioned over the neck of the sidewall aneurysm and a second portion of the cover is positioned over the entry opening of the perforating branch vessel, and wherein a median areal equivalent diameter of the pores of the first portion
and a median areal equivalent diameter of the pores of the second portion are between 3 and 9 microns.

16. The method of claim 15, wherein at least 95% of the pores by pore count of the first portion of the cover and the second portion of the cover have an areal equivalent pore diameter of less than 20 microns.

17. The method of claim 16 wherein 5% to 20% of the pores by pore count of the first portion of the cover and the second portion of the cover have an areal equivalent pore diameter of 12 or more microns.

18. The method of claim 15, wherein the first portion of the cover and the second portion of the cover consist essentially of electrospun filaments.

19. The method of claim 18, wherein a thickness of the cover over at least 70% of the total cover area is less than 7 microns.

20. An intravascular flow diverter for treating an aneurysm of a blood vessel, the intravascular flow diverter comprising:
one or more wires forming a frame, wherein the frame has a collapsed configuration and an expanded configuration, wherein the frame is configured to transition in use from the collapsed configuration to a deployed configuration that substantially conforms to a shape of an inside surface of the blood vessel in the vicinity of the aneurysm;
a cover coupled to and extending over at least a portion of the frame;
wherein the cover comprises pores therethrough over the at least a portion of the frame;
wherein in the expanded configuration a thickness of the cover over at least 70% of the total cover area is less than 7 microns;
wherein in the expanded configuration at least 95% of the pores by pore count over the at least 70% of the total cover area have an areal equivalent pore diameter of less than 20 microns;
wherein in the expanded configuration a median areal equivalent pore diameter of the pores over the at least 70% of the total cover area is between 3 and 9 microns;
wherein in the expanded configuration 10% to 70% of the pores by pore count over the at least 70% of the total cover area have an areal equivalent pore diameter of less than 5 microns;
wherein in the expanded configuration 70% to 95% of the pores by pore count over the at least 70% of the total cover area have an areal equivalent pore diameter of less than 10 microns;
wherein in the expanded configuration 5% to 30% of the pores by pore count over the at least 70% of the total cover area have an areal equivalent pore diameter of 10 microns or more; and
wherein in the expanded configuration the flow diverter surface coverage over the at least 70% of the total cover area is between 45% and 85%.
